(19) United States
(12) Patent Application Publication
(10) Pub. No.: US 2008/0050768 A1
(43) Pub. Date: Feb. 28, 2008

(54) Metabolic Monitoring Device

(75) Inventors: Alan Baron, Boulder, CO (US); Ralph Burns, Louisville, CO (US)

Correspondence Address: LATHROP & GAGE LC, 2345 GRAND AVENUE, SUITE 2800, KANSAS CITY, MO, 64108, US

(73) Assignee: TAUTHETA INSTRUMENTS LLC, Boulder, CO (US)

(21) Appl. No.: 11/843,511

(22) Filed: Aug. 22, 2007

Related U.S. Application Data
(60) Provisional application No. 60/839,542, filed on Aug. 23, 2006.

(51) Int. Cl. C12Q 1/02; C12M 1/00

(52) U.S. Cl. 435/29; 435/288.7

(57) ABSTRACT

Systems, methods and devices for the monitoring of the metabolism and the health of cultured cells are disclosed. The devices simplify the loading of cells and enable loading under sterile conditions. The devices also allow for the culture, measuring and monitoring of the metabolism of a population of cells, and in particular, the accurate measuring of metabolic states simultaneously in multiple cell populations.

### RELATED APPLICATIONS

This patent application is related to and claims priority from commonly owned U.S. Provisional Patent Application Ser. No. 60/839,542, entitled: Metabolic Monitoring Device, filed Aug. 23, 2006, the disclosure of which is incorporated by reference herein.

### GOVERNMENT SUPPORT

This patent application and U.S. Provisional Patent Application Ser. No. 60/839,542 were made with Government Support, National Science Foundation Grant No. 0611015. The Government has certain rights in these patent applications.

### TECHNICAL FIELD

The disclosed subject matter relates generally to methods and apparatus for monitoring the metabolic state and viability of living tissue or a live cell population, that has been cultured in-vitro using continuous or semi-continuous perfusion of culture media. The disclosed subject matter also relates to methods and apparatus for monitoring oxygen consumption, and key metabolic components of a live cell population, that is captive in one or more cell culture chambers.

### BACKGROUND

Metabolism is the biochemical process in living organisms, where energy, harvested from an organism's environment, is used to synthesize molecules or break down molecules into components needed to sustain life. The relative health and viability of a living organism can be determined by monitoring its metabolic rate. While specific metabolic pathways may vary significantly across organisms, in the animal kingdom, a generally useful key indicator of aerobic metabolism is the consumption of oxygen. Other indicators of metabolic state include the oxidative state of nicotinamide adenine dinucleotide (NAD+, NADH) and of cytochrome-c, key metabolic components performing electron transfer.

The monitoring of oxygen consumption and other metabolic components is important to understanding the basic biology of cell life-cycles and the relative health of a population of cells. There has been significant work attempting to monitor the rate of metabolism of cultured cells.

An example of the importance of metabolic monitoring is in the treatment of diabetes mellitus, such as Type I diabetes. Type I diabetes is such that the body can not control glucose due to loss of beta cells, one of the components of cell clusters known as the Islets of Langerhans (hereinafter, also referred to as “Islets”). These specialized cell clusters are located in the pancreas and regulate physiological blood sugar levels by producing insulin. A transplantation of human Islets from a non-diabetic donor into a diabetic recipient, to treat diabetes was disclosed in, Hatipoglu, et al., “Islets Transplantation: Current Status and Future Directions,” Current Diabetes Reports, Vol. 5, pages 311-316 (2005).

The increasing demand for Islets of Langerhans transplantations has created the need to predict the viability of Islets of Langerhans in-vitro, before transplantation. These predictions are necessary to improve success rates and reduce the costs of this expensive procedure, as discussed in, Ichii, et al., “A Novel Method for the Assessment of Cellular Composition and Beta-Cell Viability in Human Islet Preparations,” American Journal of Transplantation, Vol. 5, pp. 1635-1645 (2005). In-vitro studies require cultures of Islets of Langerhans during extended periods of time, from hours to days. Continuous vertical perfusion (“perfusion” as used herein also known as perfusion and profusion) of oxygenated media improves the outcome of these procedures.

In vertical perfusion, cultured cells are exposed to media that flows upward, or vertically, through the cells. By flowing vertically, mechanical stress on the cells due to the hydrodynamic pressure against the cells is minimized. This pressure against the cells counteracts gravity and serves to suspend the cells, for example, as disclosed in, Sweet, et al., “Continuous Measurement of Oxygen Consumption by Pancreatic Islets,” Diabetes Technology & Therapeutics, Vol. 4, No. 5, pp. 661-672 (2002) (hereinafter “Sweet, et al.”). With continuous perfusion, cell wastes, including excreted compounds, are continually swept away, while nutrients and oxygen are continually renewed.

Accordingly, continuous vertical perfusion is an ideal culture environment, that better simulates in-vivo conditions than static in-vitro culture methods, as it continually replenishes the culture media. This is unlike static culture methods, where cells are suspended in Petri dishes, culture bags, or microtiter plates, with the culture medium not being replenished. Additionally Sweet, et al., disclosed that monitoring of oxygen during a perfusion culture can be used as a prediction of post-transplant viability in animals.

Continuous flow perfusion systems permit measurement of oxygen consumption of cultured cells. For example, the oxygen consumption in the Islets of Langerhans has been continuously monitored based on the assumption of a known amount of oxygen dissolved into media flowing into the perfusion chamber. The known amount was generated by equilibrating the media with a standard gas mixture. The oxygen concentration of the media was measured by the oxygen sensor at the outflow. The assumption made was that the difference between the oxygen concentration in the outflow and the expected value from the standard solution was due only to oxygen consumption of the contained Islets of Langerhans.

Conventional perfusion systems have several operational limitations which limit their utility and ease of use. For example, these conventional perfusion systems are difficult to load with cells while maintaining sterility of the system. This is because, as noted in Sweet, et al., loading cells in to the systems described therein, requires assembly of numerous parts under sterile conditions. Additionally, the chamber disclosed in Sweet, et al. requires multiple steps in the loading process.

Yet another drawback of these conventional perfusion systems is that measurements of cytochrome-c oxidative state via light absorbance are electrically noisy and inaccurate. This is due to the fact that the small number of Islets of Langerhans have a small optical density. While Sweet et al., discloses light scattering beads to improve absorption, the resultant absorbance signal remains weak.

### SUMMARY

The disclosed subject matter provides systems, methods and devices for the monitoring of the metabolism and the health of cultured cells. The disclosed subject matter includes devices that simplify the loading of cells and enable loading under sterile conditions. The disclosed subject matter includes devices, to culture, measure and monitor the metabolism of a population of cells, and in particular, to accurately measure metabolic states simultaneously in multiple cell populations. By being able to measure and monitor the metabolism of cells, the disclosed subject matter may lead to higher transplantation success rates at lower costs, more efficient qualification of drug candidates prior to animal studies, better management of diabetes mellitus, and improved human health.

In one embodiment, the disclosed subject matter provides a perfusion culture chamber for monitoring the metabolism of cells contained within a defined culture region. The perfusion chamber is formed of two pieces, movable with respect to each other, for loading cells and then returned to the initial position to enclose the cells in the perfusion chambers for perfusion analysis. Each perfusion chamber is within a flow channel, which provides an inflow and outflow for culture media, and is made from oxygen impermeable material. The perfusion chamber has two porous obstructions, plugs or frits, with pores sufficiently small to prevent cells from escaping a culture region of interest within the perfusion chamber, but with porosity sufficiently large that minimal back pressure is generated. An input oxygen sensor measures the amount of oxygen dissolved in the culture media before it reaches the culture region, and an output oxygen sensor measures the amount of oxygen dissolved in the media after leaving the culture region. The difference in dissolved oxygen can be related to the rate of oxygen consumption, provided the media flow rates are known.

Another embodiment of the disclosed subject matter provides a perfusion culture chamber for monitoring the metabolism of cells contained within a defined culture region and constructed for moving the porous obstructions out of the perfusion flow path to facilitate quick and sterile loading of cells into the culture region.

In another embodiment of the disclosed subject matter, there is provided a perfusion culture chamber for monitoring the optical properties, for example, spectral absorbance or fluorescence of compounds related to the metabolism of the cultured cell population.

The disclosed subject matter also provides methods related to the use of the perfusion culture chamber. One method is directed to monitoring the oxygen consumption of the cells in the culture region. Another method measures the oxidative state of cytochrome-c of the cells in the culture region. Yet another method combines the measurement of metabolic indicators to assess the health of a cultured cell population for various purposes, such as prediction of viability when transplanted into a recipient organism, prediction of apoptosis, or the like.

Another embodiment of the disclosed subject matter is directed to an apparatus for analyzing material, for example, cells. The apparatus includes at least a first member and a second member movable, for example, by sliding, with respect to each other. There is at least one perfusion chamber, at least a portion of the at least one perfusion chamber in the first member and at least one portion of the at least one perfusion chamber in the second member. There is also at least one loading channel at least in the first member corresponding to the at least one perfusion chamber, and the first member and the second member movable with respect to each other between at least a first position and a second position. The first position is such that the at least one perfusion chamber is formed by the portions of the at least one perfusion chamber in the first member, and the second member being aligned, to enclose a volume for holding the cells in a sealed and sterile arrangement, free of ambient contaminants. The second position is such that the at least one loading channel is operatively coupled, for example, in alignment, with the portion of the at least one perfusion chamber in the second member.

Another embodiment of the disclosed subject matter is directed to an apparatus for analyzing material. The apparatus includes at least a first member and a second member movable with respect to each other between a first position and a second position, for example, by sliding. There is an inflow port in the second member and a plurality of outflow ports in the first member. There is also a plurality of flow channels, with each flow channel of the plurality of flow channels coupled with the inflow port and an outflow port of the plurality of outflow ports. There is a perfusion chamber formed in each of the flow channels of the plurality of flow channels, and at least a portion of each perfusion chamber is in the first member and the second member. The portions of each perfusion chamber in the first member and the second member enclose a volume when the first member is in the first position with respect to the second member. There is also a plurality of loading channels in the first member, each of the loading channels of the plurality of loading channels corresponding to the perfusion chamber in each of the flow channels, the loading channels for coupling with the portion of each perfusion chamber in the second member, when the first member is in the second position with respect to the second member.

Another embodiment of the disclosed subject matter is directed to a method of material analysis, for example, analysis of cells. The method is such that there is provided an apparatus including, at least a first member and a second member movable with respect to each other, at least one inflow port, and at least one outflow port. There is at least one flow channel in operatively coupled with the at least one inflow port and the at least one outflow port. There is at least one perfusion chamber in the at least one flow channel, with at least a portion of the at least one perfusion chamber in the first member and at least one portion of the at least one perfusion chamber in the second member. There is also at least one loading channel at least in the first member corresponding to the at least one perfusion chamber. The first member and the second member are movable with respect to each other between at least a first position and a second position. The first position is such that the at least one perfusion chamber is formed by the portions of the at least one perfusion chamber in the first member and the second member being in an operative coupling, for example, alignment, with each other so as to enclose a volume. The second position is such that the at least one loading channel is operatively coupled, for example, aligned, with the portion of the at least one perfusion chamber in the second member.

The first member and the second member are then moved to the second position, and material, for example, cells in culture media (cells) are loaded into the perfusion chamber. Once the cells have been loaded, the first member and the second member are moved to the first position, such that the material, for example, the cells, are enclosed in the volume of the at least one perfusion chamber. Perfusion media is then moved through the at least one flow channel, including the at least one perfusion chamber, to perfuse the material, for example, the cells, in the perfusion chamber. Movement or perfusion of the perfusion media is in the direction from the inflow port and out of the apparatus through the outflow port. Optical analysis may be performed on the cells in the perfusion chamber and oxygen measurements, for example, oxygen concentration measurements may be made of the perfusion media at various points along its flow path through the apparatus.

### BRIEF DESCRIPTION OF THE DRAWINGS

Attention is now directed to the drawing figures, where corresponding or like numerals or characters indicate corresponding or like components. In the drawings:

FIG. 1A is a front perspective view of an apparatus of the disclosed subject matter in a first storage or perfusion position;

FIG. 1B is a rear perspective view of the apparatus of FIG. 1A;

FIG. 2 is a cross sectional view of the apparatus of FIG. 1A taken along line 2-2;

FIG. 3A is a top exploded view of the apparatus of the disclosed subject matter;

FIG. 3B is a bottom exploded view of the apparatus of the disclosed subject matter;

FIG. 4 is a cross sectional view of the apparatus of FIG. 1A taken along line 4-4;

FIG. 5 is a cross sectional view of the apparatus of FIG. 1A taken along line 5-5;

FIG. 6A is a cross sectional view of the apparatus of FIG. 1A in a storage position taken along line 6A-6A;

FIG. 6B is a cross sectional view of the apparatus of FIG. 1A in a storage position taken along line 6B-6B;

FIG. 7 is a schematic diagram of the apparatus of FIG. 1A in an exemplary operation;

FIG. 8A is a front perspective view of an apparatus of FIG. 1A in the second or loading position;

FIG. 8B is a an opposite side perspective view of the apparatus of FIG. 8A;

FIG. 8C is a cross sectional view of the apparatus of FIG. 1A in a second or loading position taken along line 6B-6B;

FIG. 9 is a cross sectional view of the apparatus of FIG. 1A in a perfusion position taken along line 6B-6B;

FIGS. 10A, 10B and 11 are schematic diagrams of example perfusion chambers undergoing optical analysis;

FIG. 12A is a schematic diagram of perfusion chambers subject to flow restriction; and,

FIG. 12B is a diagrammatic view of the apparatus of FIG. 1A subject to flow restriction in accordance with the schematic diagram of FIG. 12A.

### DETAILED DESCRIPTION OF THE DRAWINGS

In this document, references are made to directions, such as upper, lower, top, bottom, up, down, upward, downward, front, rear, forward, backward, upstream, downstream, etc. These directional references are exemplary, to show the disclosed subject matter in an example orientation, and are in no way limiting.

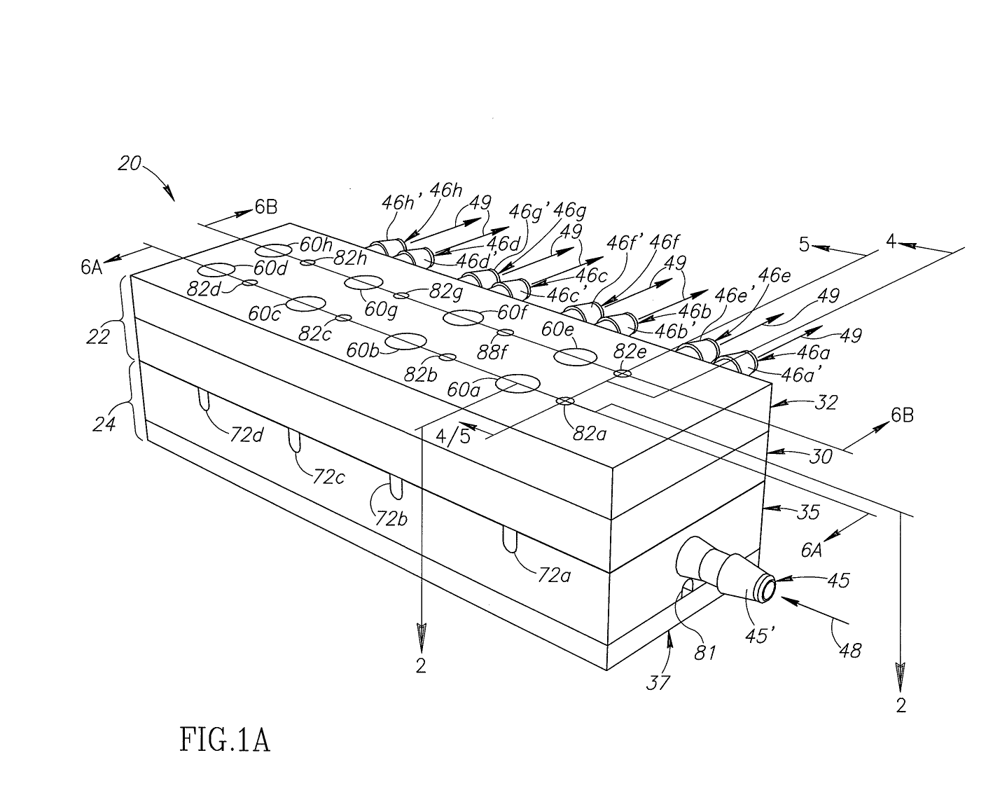

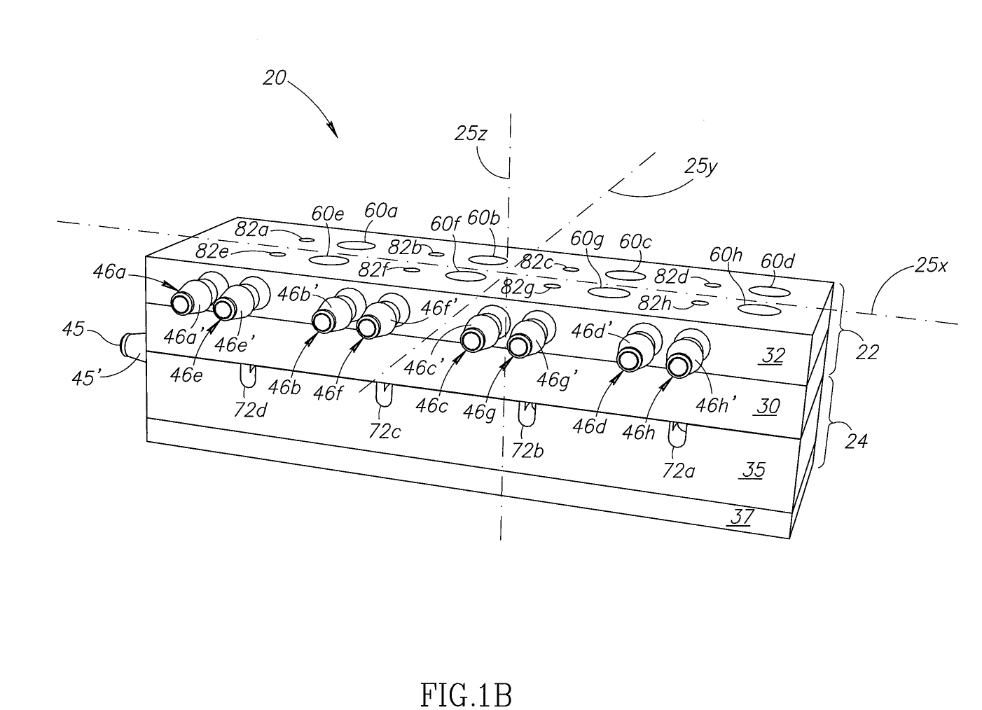

FIGS. 1A and 1B show an apparatus 20, for vertical perfusion of tissue and/or cultured cell populations. The apparatus 20 is formed of two pieces 22, 24, movable with respect to each other, for example, by sliding, a first or upper piece 22 with respect to a second or lower piece 24, and vice versa. For purposes of orientation, the apparatus includes a longitudinal axis 25x, a transverse axis 25y and orthogonal axis 25z, as shown in FIG. 1B. A first or upper piece 22 is formed by a first plate 30 and a second plate 32, while a second or lower piece 24 is formed by a first plate 35 and a second plate 37.

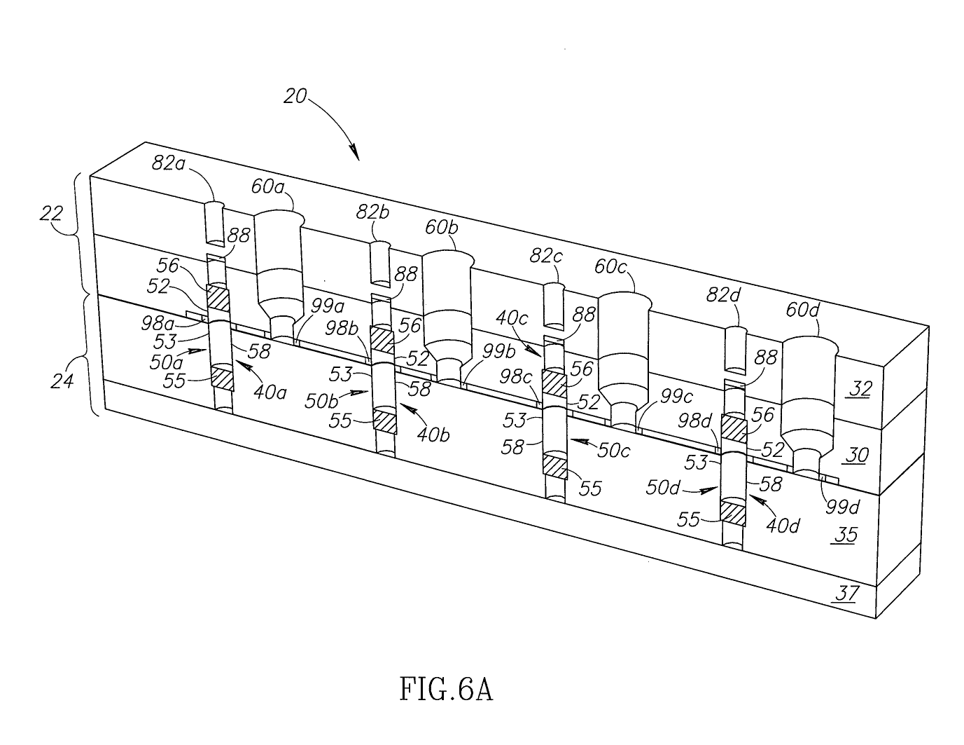

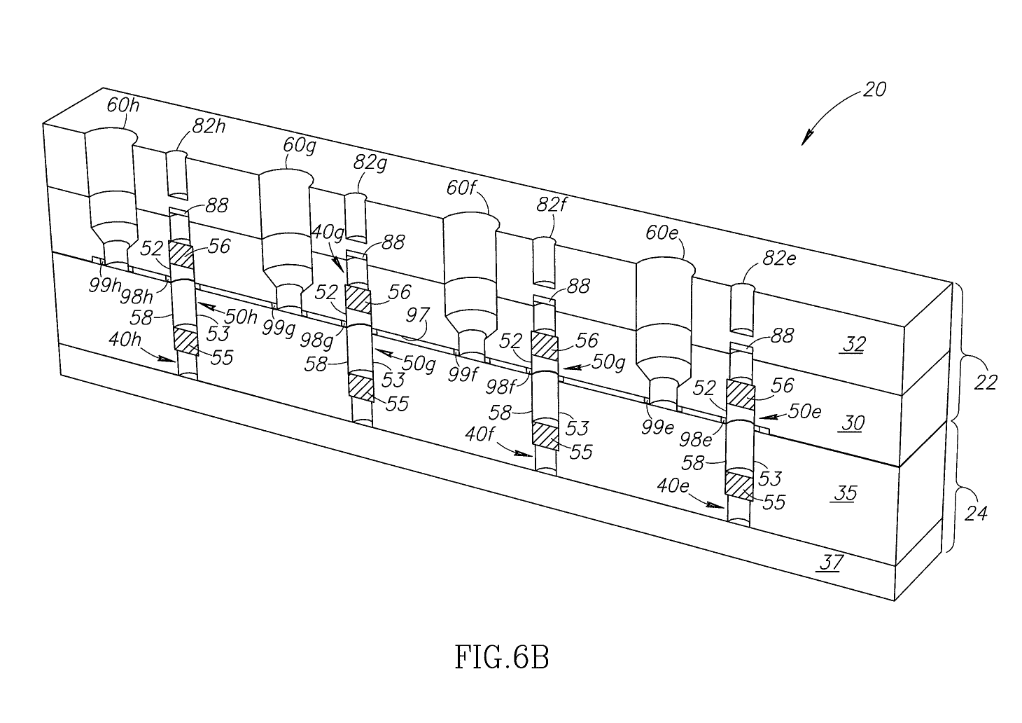

Flow channels 40a-40h (FIGS. 6A and 6B) are within the apparatus 20, and with portions thereof formed in each of the two pieces 22, 24. The flow channels 40a-40h extend, for example, from a branch line 91a-91h (via a serial line 44, an inflow line 44a, and an inflow port 45) to their respective outflow ports 46a-46h. For example, separate outflow ports 46a-46h allow for collection of media fractions for monitoring cellular insulin production or production of other proteins. There are, for example, eight flow chambers 40a-40h shown, with any number (one or more) of flow channels suitable in the apparatus 20. Flow of perfusion media or perfusate (perfusion media, perfusion flow media and perfusate used interchangeably herein) through the flow channels 40a-40h is in the direction indicated by the arrows 48 (inflow), 49 (outflow).

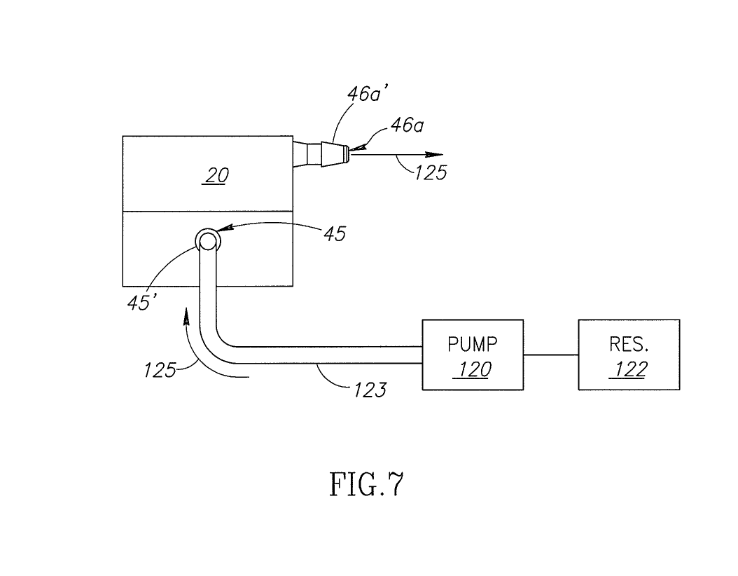

The inflow port 45 and outflow ports 46a-46h are, for example, hose barbs 45′, 46a′-46h′ or compression fittings. By being in this configuration, hose lines, tubing 123 (FIG. 7) and the like may be easily attached thereto.

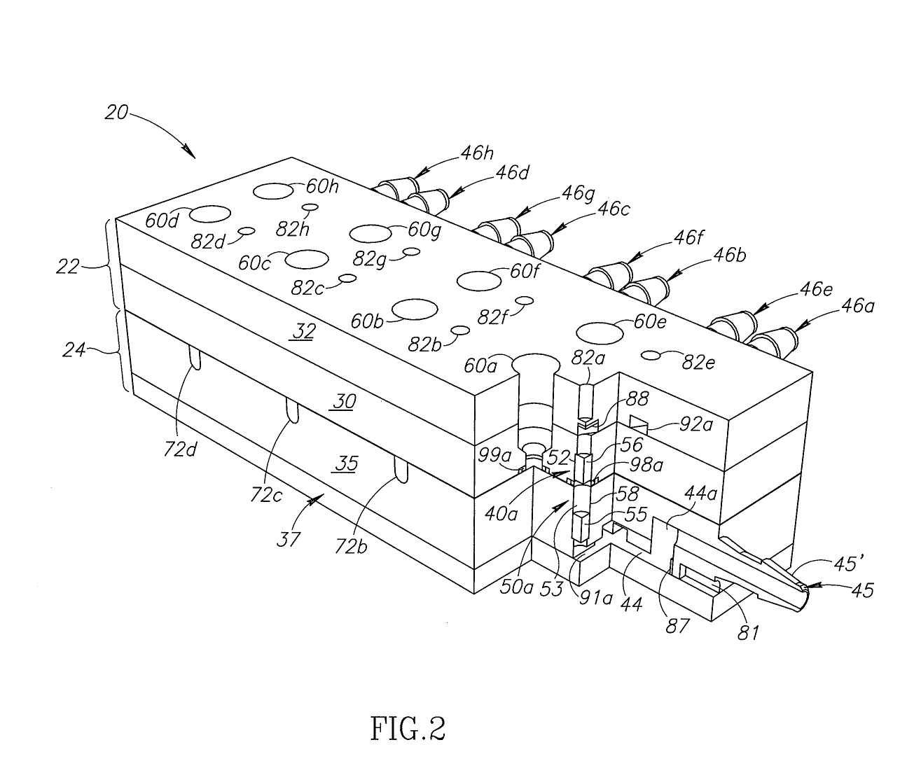

Perfusion chambers 50a-50h (FIGS. 6A and 6B), for example, eight, corresponding to each of the respective flow channels 40a-40h, are within each respective flow channel 40a-40h. Each perfusion chamber 50a-50h, for example, represented by the perfusion chamber 50a, and shown in FIG. 2, is formed by a portion 52, in the first piece 22, and a corresponding portion 53 in the second piece 24. The portions 52, 53 of each perfusion chamber 50a-50h, formed in the respective first 22 and second 24 pieces are constructed to align for media flow therethrough and through the respective flow channels 40a-40h, when the apparatus 20 is in a first, storage or perfusion position, as shown in FIGS. 1A and 1B. The actual “storage” or “perfusion” position as shown in FIGS. 1A and 1B, is dependent on whether or not the perfusion chambers 50a-50h are loaded with cells in culture media.

Each perfusion chamber 50a-50h is defined by a lower frit 55 in the second piece 24 and typically an upper frit 56 in the first piece 22. The frits 55, 56 are of a porous material, such as polyethylene. The volume (when empty) or culture region (when filled with cells, cells in culture media or the like) 58 between the frits 55, 56 is where the cells in culture media are placed and remain during perfusion with perfusate, identical or similar to the culture media. The frits 55, 56 are, for example, constructed of a porous material with voids that are fine enough to prevent the cells in the culture region or volume 58 from escaping the top of the culture region 58 due to hydrodynamic pressure, or escaping through the bottom of the culture region 58 due to gravity when the flow of perfusate is stopped.

The flow channels 40a-40h, perfusion chambers 50a-50h and frits 55, 56 are typically of a cylindrical geometry. The perfusion chambers 50a-50h, may be, for example, approximately 3 mm in diameter, to be suitable for holding cells, such as Islets of Langerhans, for analysis. Other geometries such as semi-cylindrical, rectangular and square are also suitable, provided that the flow channel 40a-40h minimizes dead or unused volume.

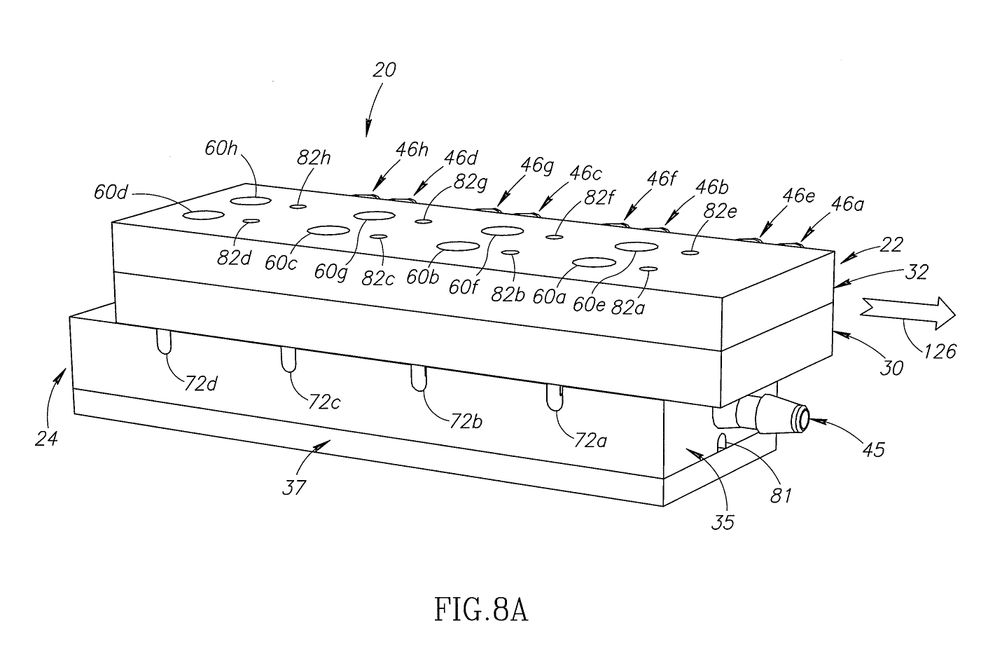

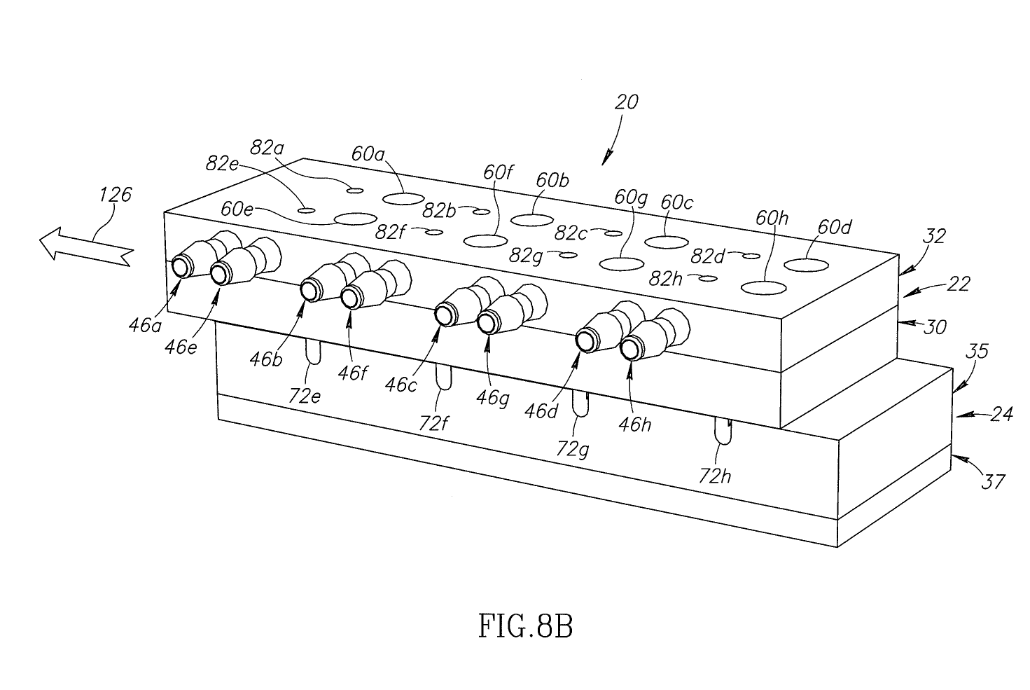

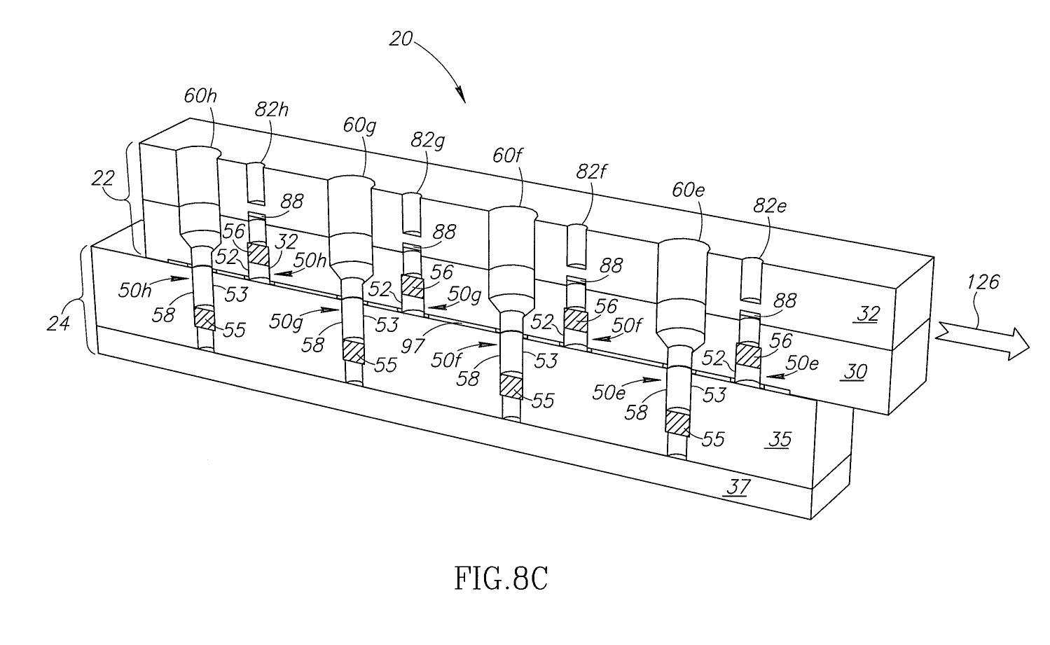

The cells 127 in culture media in the culture region 58 (FIGS. 8C, 9, 10A, 10B, 11, 12A and 12B), may be, for example, cells such as Islets of Langerhans, and other cellular material and the like. These cells are subject to perfusion in the culture region 58. For example, the volume or culture region 58 may be such that it can accommodate a culture of approximately 50-400 Islets of Langerhans. These Islets of Langerhans cells can be cultured, for example, with any suitable culture media for these kinds of cells. Additionally, for example, these Islets of Langerhans cells may be cultured in accordance with Sweet, et al., “Continuous Measurement of Oxygen Consumption by Pancreatic Islets,” Diabetes Technology & Therapeutics, Vol. 4, No. 5, pp. 661-672 (2002), this document incorporated by reference herein. From the perfusion of the cells in the perfusion chambers 50a-50h, oxygen concentrations, oxygen consumption rates, cytochrome-c, NAD+, NADH, and the like may be obtained.

Each perfusion chamber 50a-50h includes its own loading channel 60a-60h. Through the respective loading channel 60a-60h, cells are introduced into the volume 58 of the respective perfusion chamber 50a-50h (in the lower portion 53 of each perfusion chamber 50a-50h). The loading channels 60a-60h are formed in the first piece 22, and are positioned to align with the lower portion 53 of the respective perfusion chamber 50a-50h, when the apparatus 20 is in a loading position, as shown in FIGS. 8A-8C, where each of the pieces 22, 24 includes a portion extending over the other. For example, the loading channels 60a-60h may be positioned with respect to each other so as to correspond to the configuration of a serial pipette, for loading of cells by a serial pipette, or other serial transfer device.

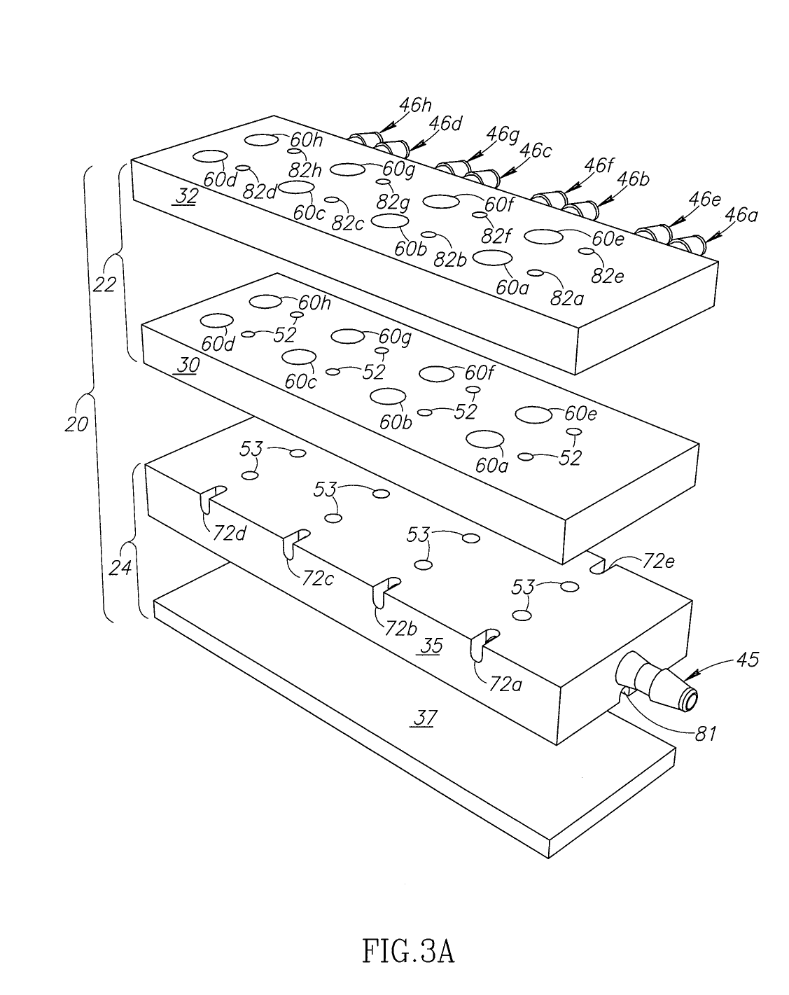

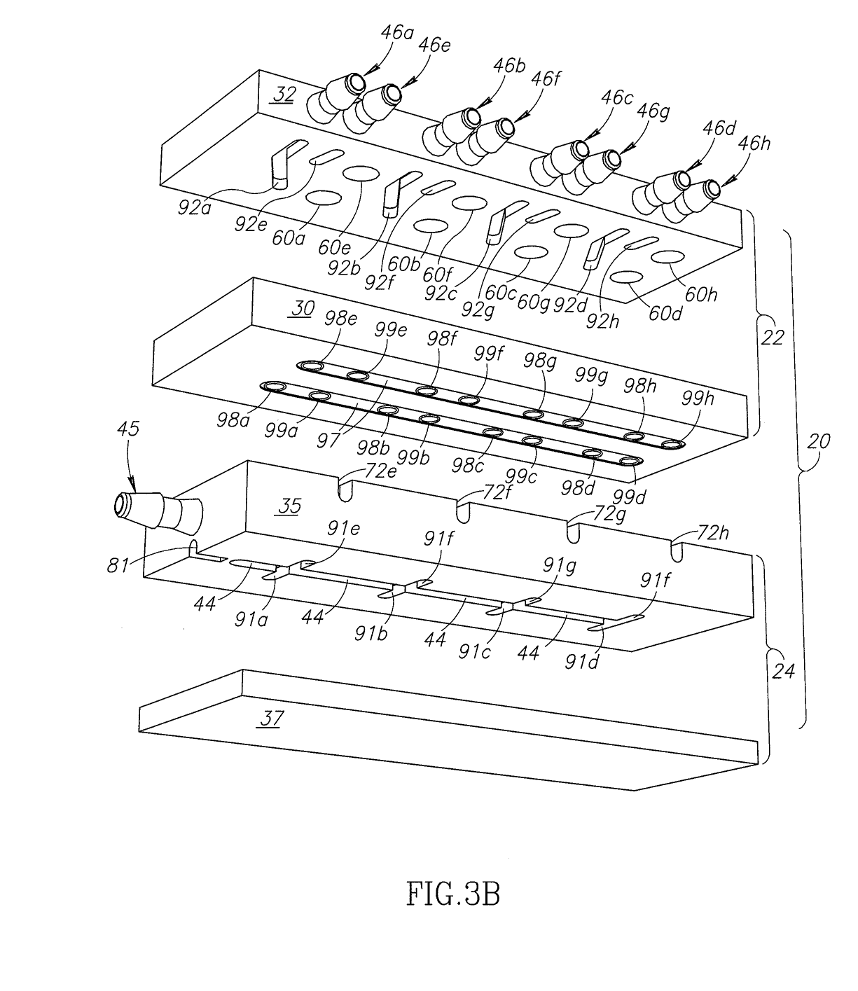

The apparatus 20 is such that each flow channel 40a-40h, and in particular, at the lower portion 53 of each perfusion chamber 50a-50h may be subjected to optical analysis, including, for example, spectral absorbance, or fluorescence. The optical analysis system includes a reflector 70 (FIGS. 2 and 3A) or other reflective or scattering background, that seats in a slot 71 in the plate 35, and corresponds to an optical port 72a-72h in the apparatus 20, for example, in the second piece 24. The reflector 70 may be, for example, white Teflon® or other similar white film.

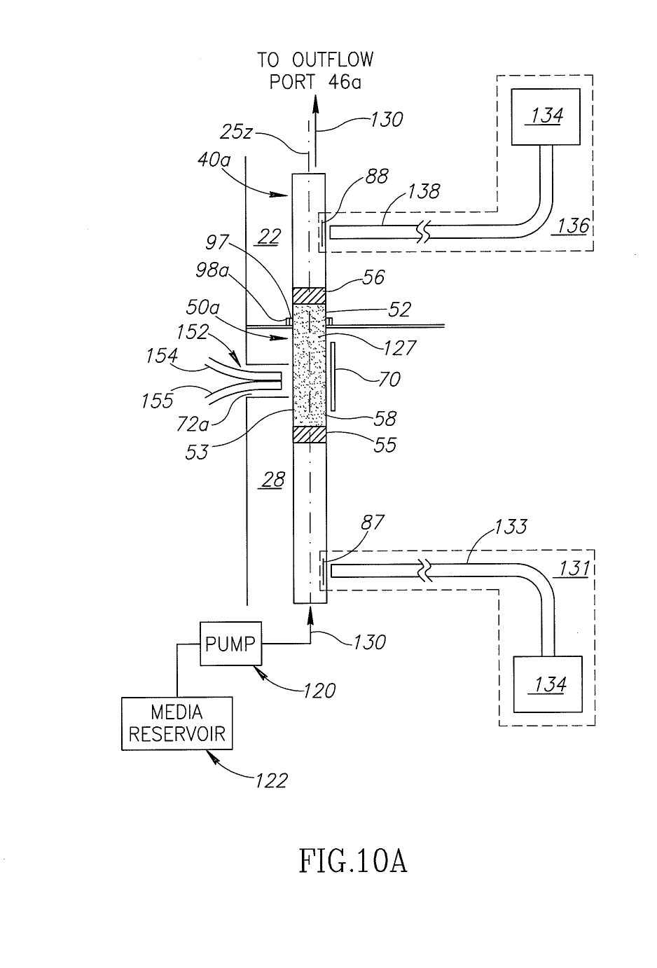

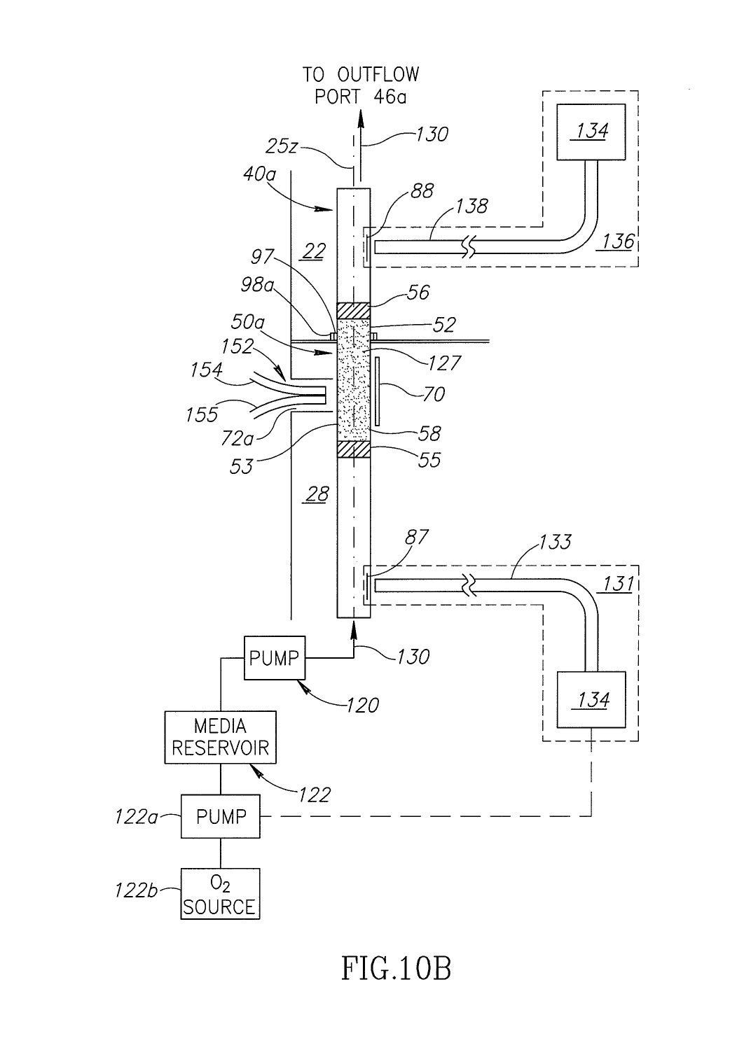

The optical ports 72a-72h, corresponding to the respective flow channels 40a-40h and perfusion chambers 50a-50h, are bores for receiving, for example, one or more optical fibers 154, 155 (FIGS. 10A and 10B), and is positioned opposite to the reflector 70, such that the energy, for example, light, transmitted from an optical fiber (for example, fiber 154 of FIGS. 10A and 10B), travels to the reflector in a direction at transverse, or substantially transverse, to the orthogonal axis 25z (FIGS. 1B, 10A and 10B) of the perfusion chamber 50a-50h, with the reflection off of the reflector 70 also traveling to the optical fiber (for example, fiber 155 of FIGS. 10A and 10B) in the aforementioned transverse or substantially transverse direction. This orientation of the optical fibers 154, 155 and reflector 70 allows for a double path length for the emitted light and single face access to the optical fibers 154, 155, through the respective optical port 72a-72h.

The fiber optics (optical fibers 154, 155 of FIGS. 10A and 10B) and their respective reflector 70 when coupled, serve in monitoring the metabolism and health of the cultured cells in the perfusion chamber 50a-50h. For example, each optical port 72a-72h may be approximately 2 mm in diameter to accommodate the requisite optical fibers 154, 155 (FIGS. 10A and 10B).

The apparatus 20 accommodates oxygen sensing systems for sensing inflow and outflow oxygen concentrations in the perfusion flow media. The apparatus 20 includes ports 81, 82a-82h proximate to the points of inflow and outflow of the perfusion media. The ports 81, 82a-82h (corresponding to each flow channel 40a-40h) are, for example, cylindrical bores extending from the surface of the apparatus 20. Oxygen sensors 87, 88 are placed proximate to the ports 81, 82a-82h. The oxygen sensors 87, 88 are, for example, formed of oxygen sensitive luminescent or fluorescent material.

The ports 81, 82a-82h, support optical fibers, for example, fibers 133, 138 (FIGS. 10A, 10B, 11 and 12A), respectively. The optical fibers 133, 138 couple the oxygen sensors 87, 88 with an analytic instrument 134 (FIGS. 10A, 10B, 11 and 12A), for detecting oxygen concentrations, from which various analyses including oxygen consumption, of the cells under analysis may be performed. For example, ports 81, 82a-82h may be approximately 2 mm in diameter to accommodate the requisite optical fibers.

The apparatus 20 supports inflow 131 and outflow 136 oxygen sensor units (FIGS. 10A and 10B). The inflow oxygen sensor unit 131 is positioned at the single port 81 in the second piece 24, in the inflow path, where inflow oxygen is sensed. This port 81 is positioned along the inflow line 44a, downstream from the inlet port 45 and upstream of the serial line 44 and perfusion chambers 50a-50h. The outflow oxygen sensor units 136 are positioned at each of the ports 82a-82h of the first piece 22, in the outflow path in each of the flow channels 40a-40h, and downstream of the perfusion chambers 50a-50h.

The flow channels 40a-40h at the perfusion chambers 50a-50h, are for example, of a width or diameter of approximately 1 mm to 5 mm, and a height of approximately 3 mm to 10 mm, with resulting volumes ranging from approximately 3 micro liters (μl) to 250 μl. Perfusion media flow rates through the flow channels 40a-40h may be, for example, from approximately 5 μl/min to 1000 μl/min.

Attention is now directed to FIG. 3B, which shows the channeling that defines the inflow path for perfusion media that leads into the flow channels 40a-40h, and ultimately through the perfusion chambers 50a-50h. Plate 35 includes the inflow line 44a, that is, for example, an “L” shaped bore, that extends from the inflow port 45 to a serial line 44. Branch lines 91a-91h, connect the serial line 44 to each flow channel 40a-40h, upstream of the respective perfusion chambers 50a-50h. Plate 37 is attached to the plate 35, enclosing the serial line 44 and branch lines 91a-91h, and providing a watertight seal for the apparatus 20.

The apparatus 20 is symmetric about is longitudinal axis 25x with respect to portions of the flow channels 40a-40h, the perfusion chambers 50a-50h, loading channels 60a-60h, reflectors 70, inflow port 45 and inflow line 44a, optical 72a-72h and oxygen sensing 81, 82a-82h ports, serial line 44 and branch lines 91a-91h. Accordingly, the discussion for one example flow channel 40a and corresponding perfusion chamber 50a is applicable to all flow channels 40a-40h, perfusion chambers 50a-50h, and loading channels 60a-60h.

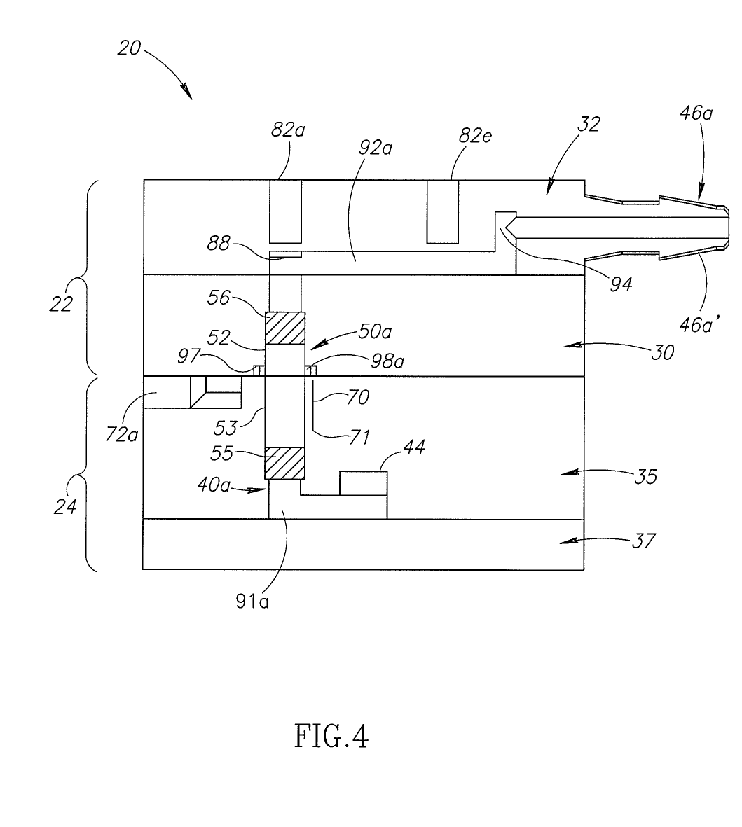

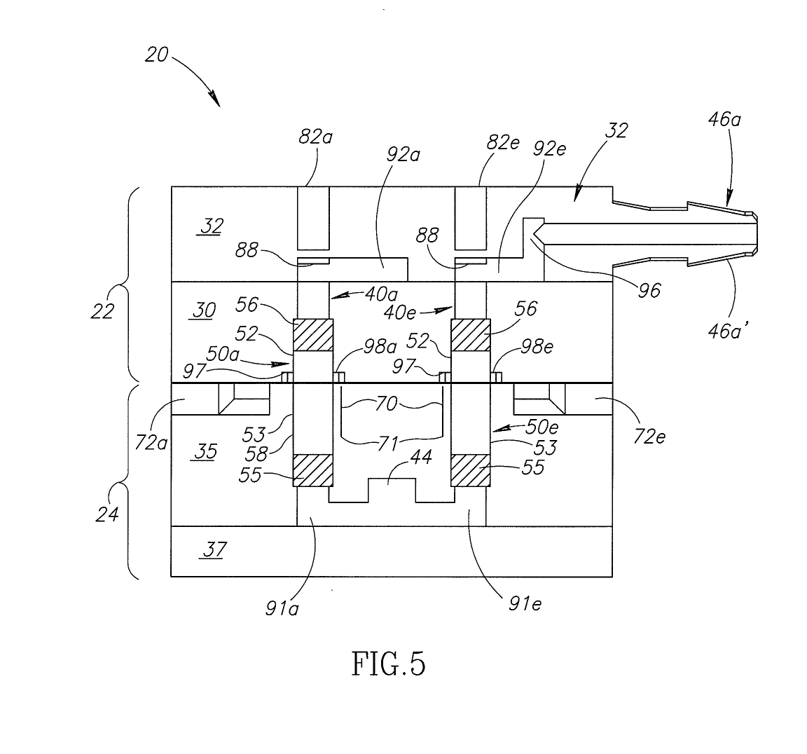

Turning also to FIGS. 3B, 4 and 5, the flow channels 40a-40d downstream of the perfusion chambers 50a-50d, terminate in the outflow ports 46a-46d. These flow channels 40a-40d are formed of grooves 92a-92d (FIG. 3B) that extend along the plate 32, and terminate in an “L” shaped bore 94, as shown in an exemplary flow channel 40a (FIG. 4). The “L” shaped bores 94 terminate in the respective outflow ports 46a-46d. Similarly, the flow channels 40e-40h downstream of the perfusion chambers 50e-50h, that terminate in the outflow ports 46e-46h, are formed of grooves 92e-92h that extend along the plate 30. The grooves 92e-92h terminate in an “L” shaped bore 96, as shown in an exemplary flow channel 40e (FIG. 5). The “L” shaped bores 96 terminate in the respective outflow ports 46e-46h. All of the grooves 92a-92h are enclosed by the attachment of the plates 30, 32, so as define the flow channels 40a-40h downstream of the perfusion chambers 50a-50h.

The plate 30, for example, as shown in FIG. 3B, includes recessed areas 97, where rounded protrusions or gaskets 98a-98h, for example, made of Teflon®, rubber or other hydrophobic material, for the upper portions 52 of the perfusion chambers 50a-50h extend from the surface of the recessed area 97. Rounded protrusions or gaskets 99a-99h, for example, made of Teflon®, rubber, or other hydrophobic material, for the loading channels 60a-60h extend from the surface of the recessed areas 97. This construction decreases the surface area for sliding and allows for less friction when the pieces 22, 24 are moved (for example, slid) between positions for loading and perfusion and storage. It also permits increased pressure for sealing and accordingly, a tighter contact between the pieces 22, 24, at the respective upper portions 52 of the perfusion chambers 50a-50h and the respective loading channels 60a-60h, and the lower portions 53 of the perfusion chambers 50a-50h in the second piece 24. It also creates a watertight seal between the pieces 22, 24, and allows them to slide while in contact.

The plates 30, 32 and 35, of the first 22 and second 24 pieces, respectively, are made of a clear or translucent material, for example, a transparent material such as clear and transparent plastic or clear and transparent glass. This transparency supports the optical properties of the reflector 70 and corresponding optical fibers as well as the oxygen sensors 87, 88 and corresponding optical fibers of the oxygen sensor units 131, 136 (FIGS. 10, 11 and 12A) Additionally, the plastic or glass is such that it allows for minimal, if any, oxygen permeability, to prevent the diffusion of oxygen in or out of the perfusion media, so as not to cause false measurements of oxygen consumption.

The other plate 37 is typically also made of the same material as plates 30, 32 and 35, respectively. Alternately, this plate 37 could be made of another plastic or other material, and need not be transparent. The plates 30, 32 and 35, 37, are adhered together by adhesives, welds and other conventional fastening techniques and the plates 30, 32, 35, 37 may be made by techniques such as injection molding, or the like, if plastic, and conventional glass-making techniques, if glass.

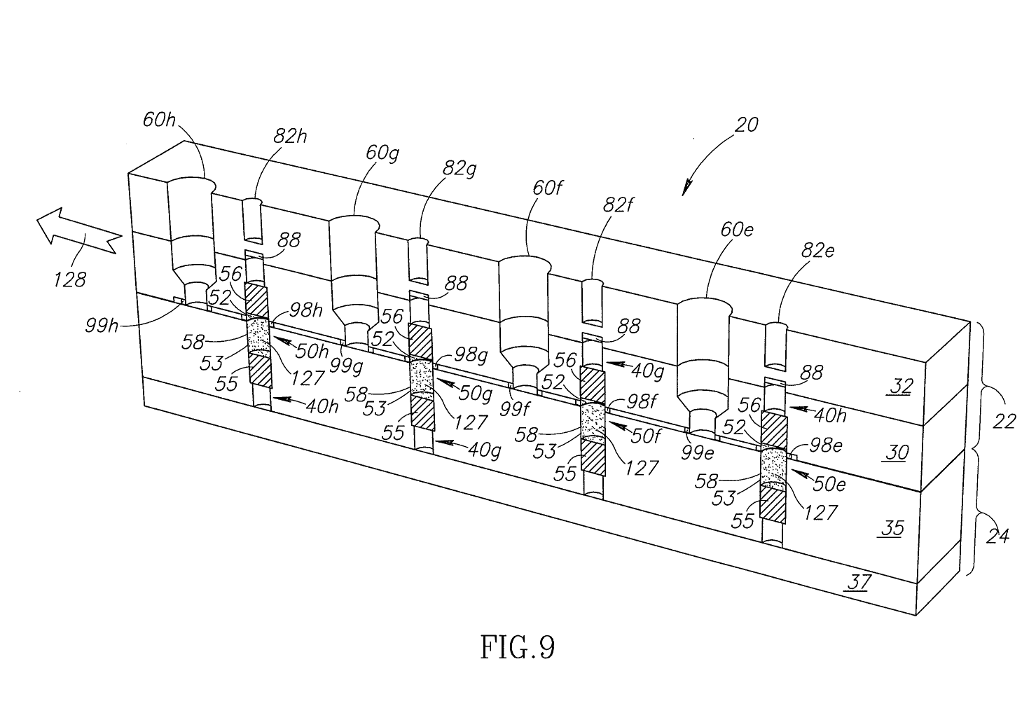

Attention is now directed to FIGS. 6A, 6B, 7, 8A-8C and 9. These figures show the two positions of the apparatus 20, a storage (FIGS. 6A and 6B) or perfusion position (FIG. 9) where the upper 22 and lower 24 pieces are coaxial (along axis 25y), and another position, a loading position (FIGS. 8A-8C), where a portion of the upper piece 22 extends beyond the lower piece 24, and vice versa.

In FIGS. 6A and 6B, the perfusion chambers 50a-50h are empty (free of cells), and the upper portions 52 are aligned with the lower portions 53 of each perfusion chamber 50a-50h. The apparatus 20 may now be connected to a pump 120, that is in turn connected to a media reservoir 122 of perfusion media, for example, RPMI medium 1640 containing 10% FBS and 3 mM glucose equilibrated with either 5% CO₂/balance air or 5% CO₂/balance nitrogen, as disclosed in Sweet, et al., or any other suitable perfusion media. This is shown schematically in FIG. 7. When activated, the pump 120 will provide flow of perfusion media through the inflow port 45, through all of the flow channels 40a-40h, that include the perfusion chambers 50a-50h and out through the respect outlet ports 48a-48h, to prime the flow channels 40a-40h of the apparatus 20, and to immerse the oxygen sensors 87, 88 in liquid. The pump 120 is, for example, programmable, and is such that it can pump in either a continuous or intermittent mode, so as to move perfusion media through the apparatus either continuously or intermittently, or combinations thereof, depending on how the pump 120 is programmed. This priming perfusion is prior to the perfusion detailed below, once the cells have been loaded.

When it is desired to load the perfusion chambers 50a-50h of the apparatus 20 with cells, the upper piece 22 is moved in the direction of the arrow 126, to move from the storage position to the loading position. In an exemplary operation, just prior to moving to the loading position, the flow channels 40a-40h and perfusion chambers 50a-50h are primed with liquid, for example, perfusion media, to avoid the flow channels 40a-40h and perfusion chambers 50a-50h being exposed to air and other ambient contaminants.

Movement to the loading position is, for example, by the upper piece 22 sliding over the lower piece 24, until the loading channels 60a-60h align with lower portions 53 of the perfusion chambers 50a-50h, in order for cultured cells to be placed therein. This alignment is shown in the cross sectional view of FIG. 8C (as the apparatus 20 is symmetric for the perfusion chambers 50a-50d and the corresponding loading channels 60a-60d, this alignment would be the same for these structures). Cells are loaded, for example, by placing a pipette end or serial pipette ends over the loading channels 60a-60h, and transferring the cells from the pipette (not shown), through the respective loading channels 60a-60h into the perfusion chambers 50a-50h. The cells may be, for example, Islets of Langerhans cells in a culture media, or other cells in culture media, for analysis.

With the cells 127 loaded in the lower portion 53 of the respective perfusion chambers 50a-50h, the upper piece 22, is then returned to its original position (in the direction of the arrow 128). The upper 52 and lower 53 portions of the perfusion chambers 50a-50h return to being aligned, and the perfusion chambers 50a-50h are now loaded with cells 127 and sealed. The perfusion chambers 50a-50h in the apparatus 20 are now in the perfusion position, as shown in FIG. 9. The lower 55 and upper 56 frits confine the cells in the culture region 58 therebetween. This arrangement has placed the cells 127 into the perfusion chambers 50a-50h in a highly sterile manner. The cell population (cells 127) in the culture region 58 is now ready for analysis or other analytical processes, for example, by perfusion, as shown in FIG. 7.

The flow of perfusate may again be activated, so as to move through the inlet port 45, through the perfusion chambers 50a-50h, and through the respective outlet ports 46a-46h. The oxygen sensors 87, 88 are immersed in liquid (perfusion media) when the oxygen measurements are taken. Exemplary oxygen and optical measurements are shown in detail in FIGS. 10-12, and discussed immediately below.

Returning also to FIG. 7, perfusion of the apparatus 20 (and the perfusion chambers 50a-50h) is such that the media reservoir 122 contains perfusion media that is appropriate to the type of cells in the cell population under study, and is, for example, similar or identical to the culture media for the cells in the culture region 58. For example, Islets of Langerhans cells are in media, as detailed above. The pump 120 forces media through the inflow port 45 (the pump 120 connected to the inflow port hose barb 45′ by tubing 123 or the like), into the inflow line 44a, through the serial line 44 and branch lines 91a-91h and into the flow channels 40a-40h, and through the perfusion chambers 50a-50h, (in the direction of the arrows 128). The pump 120 is designed to provide a steady flow rate of media through the flow channels 40a-40h including the perfusion chambers 50a-50h. Perfusion media flows out of the apparatus 20 through the outflow ports 46a-46h, represented by the outflow port 46a.

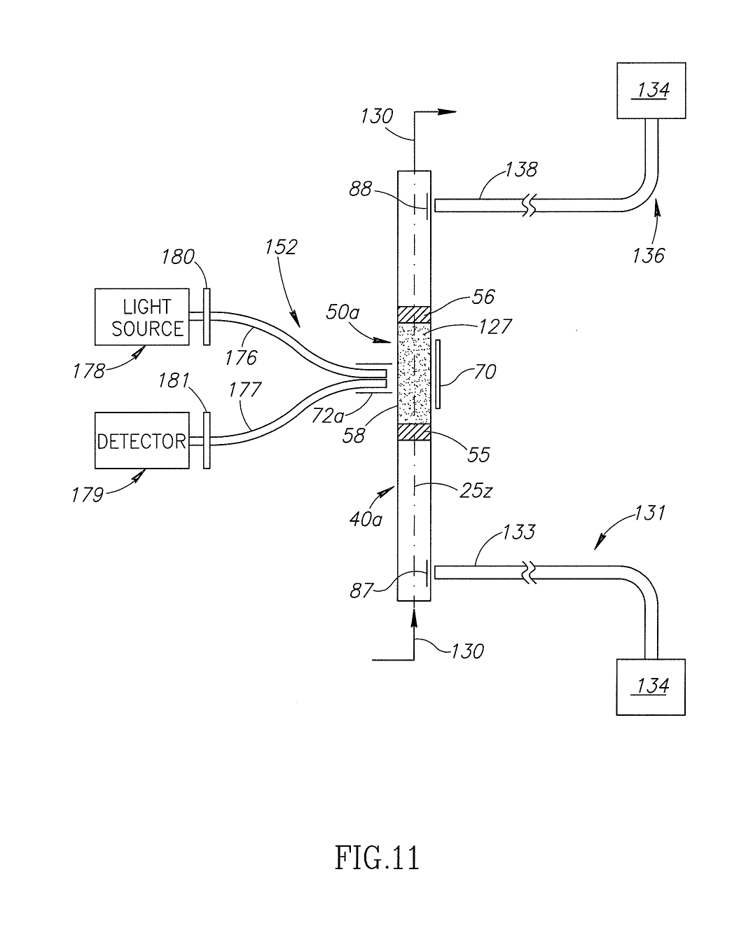

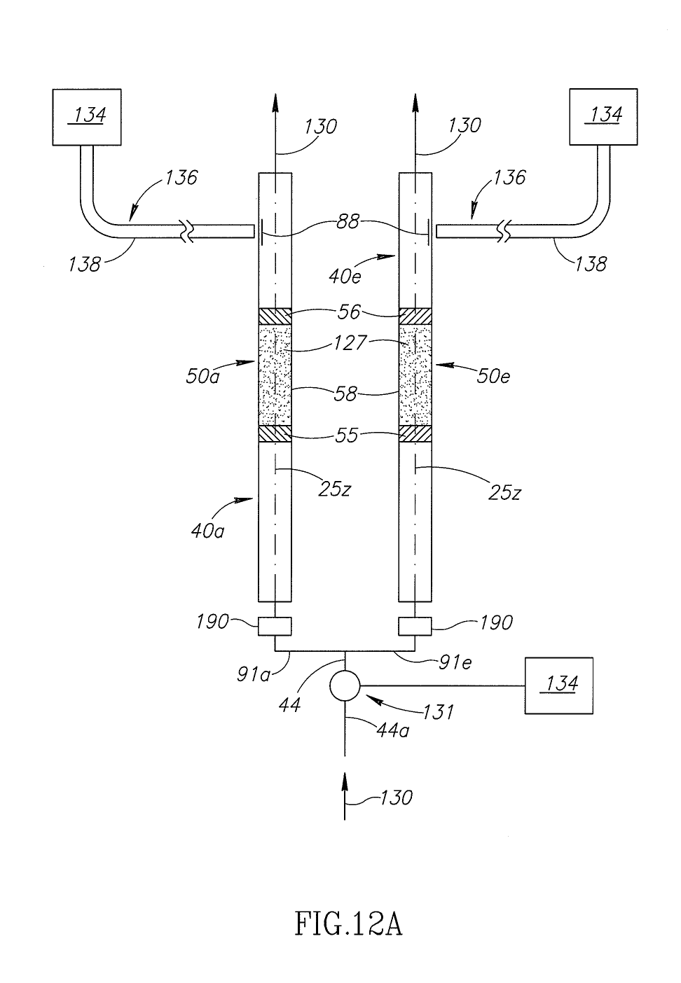

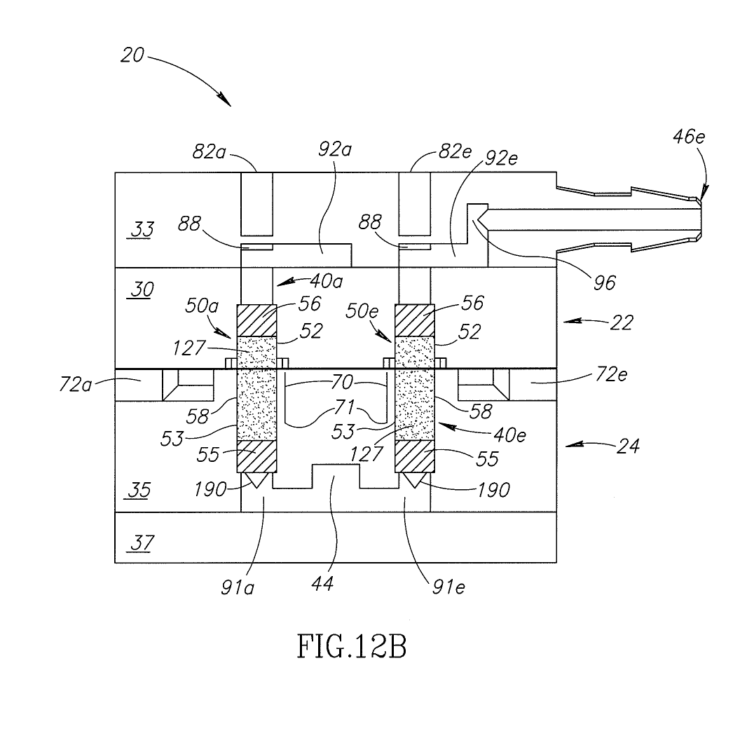

Attention is now directed to FIGS. 10A, 10B, 11, 12A and 12B, that show exemplary perfusion chambers 50a (FIGS. 10A, 10B and 11), and 50a and 50e (FIGS. 12A and 12B), as representative of all perfusion chambers 50a-50e of the apparatus 20. Accordingly, similar components and directional arrows for perfusion media flow have the same numbering and are applicable for all of these figures, except where differences are indicated.

Referring to FIG. 10A, an embodiment of the apparatus 20 with perfusion chambers 50a-50h, represented, for example, by the schematic diagram of perfusion chamber 50a in the flow channel 40a (representative of the flow channels 40a-40h), is shown. The flow channel 40a receives perfusion media from the serial line 44, through the inflow port 45 (from the pump 120 and reservoir 122). The frits 55, 56 serve to confine the cells in the volume 58. Media flow through the flow channel 40a and the perfusion chamber 50a is the direction of the arrows 130.

Alternately, only one frit 55 is necessary when the flow rate of the perfusion media is chosen to be sufficiently low, for example, approximately 1 μl/min to 25 μl/min. This low flow rate does not cause the cells in the culture region 58 to escape downstream (toward the outflow ports 46a-46e).

An inflow oxygen sensor unit 131 (shown for emphasis only in the broken line box in FIG. 10A), formed of a oxygen sensor 87, and an optical fiber(s) 133, that couples the oxygen sensor 87 to an analytic instrument 134, that analyzes data obtained from the oxygen sensor 87. The oxygen sensor 87 is positioned on the inside wall of the inflow line 44a (FIG. 2), upstream of the perfusion chamber 50a, proximate to the inflow port 46. The inflow oxygen sensor unit 131 measures the oxygen concentration of the perfusion media flowing into the culture region 58.

Outflow oxygen sensor units 136 (shown for emphasis only in the broken line box in FIG. 10A) monitor the outflow oxygen concentration from the respective flow channels 40a-40h. Each outflow oxygen sensor unit 136 is formed of an oxygen sensor 88, and an optical fiber(s) 138, that couples the oxygen sensor 88 to an analytic instrument 134, that analyzes data obtained from the oxygen sensor 88. The oxygen sensor 88 is similar to the oxygen sensor 87 detailed above, and discussed further below, as is the optical fiber(s) 138 to the optical fiber(s) 133 detailed above, and discussed further below. The oxygen sensors 88 are positioned inside the respective flow channel 40a-40e, downstream of the respective perfusion chamber 50a-50e.

The oxygen sensor units 131, 136, through their respective analytic instruments 134, measure the oxygen concentration (saturation) of the perfusion media entering (inflow unit 131) and leaving (outflow unit 136) the culture region 58. These oxygen sensor units 131, 136 are positioned in this manner, so that the oxygen concentration in the media in each perfusion chamber 50a-50h does not change due to diffusion of oxygen in or out of the cell culture media. Depending on materials used for media handling, flow rates, oxygen saturation and temperature, the amount of oxygen adsorption or desorption from materials carrying the media will change, and the necessary distance from the oxygen sensor to the culture region 58 will depend on these experimental parameters. Moreover, the oxygen consumption in each of the perfusion chambers 50a-50e is a function of the difference between the oxygen concentrations measured by the inflow sensor unit 131 and the outflow sensor unit 136.

The oxygen sensors 87, 88, are, for example, luminescent sensors, also known as fluorescent sensors. Luminescent sensors have an oxygen quenchable luminescent molecule dispersed in an oxygen permeable matrix. When the luminescence molecule containing matrix is exposed to culture media, the luminescence intensity or decay lifetime is inversely proportional to the concentration of oxygen contained in that media. Luminescent oxygen sensors may be extremely small and unobtrusive. The oxygen sensors 87, 88, when luminescent sensors, reside entirely inside the flow channels 40a-40h and are interrogated from the outside of the flow channels 40a-40h with an optical fiber 133,138 or other light transceiver. The optical fibers 133, 138 or other light transceiver, transmit and receive light from the oxygen sensors 87, 88 and the analytic instrument 134.

The analytic instruments 134 are programmed to determine, for example, oxygen concentrations, levels, amounts, etc. Alternately, the instruments 134 may be linked (electronically) to a computer (not shown) to perform the aforementioned functions.

Additional suitable exemplary oxygen sensor units are described, for example, in Sweet et al., “Continuous Measurement of Oxygen Consumption by Pancreatic Islets”, in Diabetes Technology & Therapeutics, Vol. 4, No. 5, pp. 661-672 (2002), and Wolfbeis, “Materials for Fluorescence-Based Chemical Sensors,” Journal of Material Chemistry, Vol. 15, pp. 2657-2669 (2005), both of these documents are incorporated by reference herein. Commercially available oxygen sensor units, such as the MFPF 100 from TauTheta Instruments, LLC, Boulder, Colo. 80301, USA, are also suitable.

An optical interface at or proximate to the culture region 58 serves to measure absorbance and fluorescence properties of the cultured cells 127. As also illustrated in FIG. 10A, the optical interface includes an optical fiber assembly 152 opposite a reflector 70 placed across the culture region 58. For example, the optical fibers 154, 155 of the optical fiber assembly project (fiber 154) and receive (fiber 155) energy, for example, white light, in a direction perpendicular or substantially perpendicular to the orthogonal axis 25z of the flow channel 40a and perfusion chamber 50a. This configuration is also referred to as epi-axially.

The optical fibers 154, 155 couple with instruments (not shown), that may be programmed to determine, for example, absorbance spectrometry, from which the state of cytochrome-c may be determined. Alternately, the instruments may be linked to a computer (not shown) to perform the aforementioned functions.

FIG. 10B is similar to FIG. 10A, except it shows the media reservoir 122 including an oxygen pump 122a that controls oxygen flow into the reservoir 122, from an oxygen source 122b, such as an oxygen tank. The oxygen sensor unit 131 for the inflow oxygen is such that the instrument 134 is electronically linked to the oxygen pump 122a, to serve as a feedback control for oxygen concentration in the perfusion media (in the media reservoir 122).

FIG. 11 shows an alternate optical fiber assembly 152′ for the flow channel 40a and perfusion chamber 50a in detail. Flow of perfusion media (perfusate) is in accordance with the direction of the arrows 130. For example, the fiber assembly 152′ is in two branches, a source branch 176, and a detect branch 177. A light source 178 projects Ultraviolet (UV), visible, near infrared or infrared light or radiation into the source branch 176. The source branch 176 provides light for either an absorbance or fluorescence measurement. The detect branch 177 collects light from the culture region 58 and transmits the light to a detector 179. The light from the light source 178 is selectively passed by a filter 180, and similarly, the light received in the detector 179 (through the detect branch 177) is also selectively passed by a filter 181.

For absorbance measurements, the light source 178 functions as a broadband light source, emitting wavelengths and intensities appropriate for the intended measurement. The detector 179 is configured to record light intensity as a function of wavelength. For fluorescence measurements, for example, the light source 178 output wavelengths typically match the absorbance wavelengths of the compound, cell, molecule, component or compound of interest.

Depending on whether the absorbance or fluorescence of the culture region 58 is to be monitored, the detector 179 may function in a variety of modes. For absorbance or fluorescence measurements, the detector 179 records light intensity as a function of wavelength over a broad range, for example from approximately 200 nanometers (nm) to 1200 nm.

The detector 179, may be, for example, a Model 2000 fiber optic spectrometer, available from Ocean Optics Inc, Dunedin, Fla. Alternatively, a simplified detector comprising a single light sensitive element, such as a photodiode, or photomultiplier tube, could be used in conjunction with wavelength selective optical filters.

The detector 179 may be programmed to determine, for example, for absorbance or fluorescence, for example, to determine the state of cytochrome-c with absorbance, or for, example, to identify compounds, cells, molecules, components, or compounds of interest, such as NAD+ and NADH, with fluorescence. Alternately, the detector may be linked to a computer (not shown) to perform the aforementioned functions.

Alternate configurations of source branch 176 and detect branch 177 fiber optics may be used. These alternate configurations should be such that they maximize signal return and minimize complexity in a diffuse reflectance arrangement, such as that shown in FIG. 11. In the diffuse reflectance arrangement, the excitation and detection takes place nearly epi-axially, either through a single fiber or through two parallel and substantially adjacent fibers. The reflector 70 serves to return light that has traveled across the culture region 58 back towards the detect branch 177 and enhances the absorption path length and return of fluorescence intensity. The reflector 70 enhances the sensitivity of fluorescence and absorbance measurements made on the culture region 58.

If desired, one or more optical fiber assemblies 152, 152′ may be combined in any combination. This allows for simultaneous absorbance and fluorescence measurements.

FIGS. 12A and 12B show structure similar to that of FIGS. 10A, 10B and 11 detailed above, except that the flow channels 40a-40h and perfusion chambers 50a-50h, represented by flow channels 40a and 40e, with corresponding perfusion chambers 50a and 50e, include flow restrictors 190. The flow restrictors 190 are, for example, small orifices or restrictor bores of approximately 0.1 mm to 0.5 mm. Flow of perfusion media (perfusate) is in accordance with the direction of the arrows 130. The structure of FIGS. 12A and 12B is such that it may employ either of the optical systems 152, 152′ detailed above.

In this apparatus with the flow restrictors 190, oxygen consumption measurements are made in each perfusion chamber 50a, 50e by comparing the difference in oxygen measured at the single inflow oxygen sensor 131, and the multiple outflow oxygen sensors 136, that measure oxygen concentration in the perfusate outflow from each flow channel 40a, 40e. The inflow oxygen sensor 131 may be used provided that the flow rate of media through the two perfusion chambers 50a, 50e does not substantially differ (for example, on the order of approximately ±10%. Since inconsistencies in the flow channels 40a, 40e and in the frits 55, 56 may cause different flow rates through the perfusion chambers 50a, 50e, a flow restrictor 190, introduced before each flow channel 40a, 40e (for example, in the respective branch line 91a, 91e) of the respective perfusion chamber 50a, 50e, may be used to equalize flows through the individual flow channels 40a, 40e.

Other useful features and enhancements to the disclosed subject matter include the use of temperature regulation by placing the device in a controlled temperature bath or oven, and the monitoring of the metabolic response of cells to the addition of various kinds of media, drugs or agents that stimulate or suppress cellular function.

While preferred embodiments of the disclosed subject matter have been described, so as to enable one of skill in the art to practice the disclosed subject matter, the preceding description is intended to be exemplary only. It should not be used to limit the scope of the disclosure, which should be determined by reference to the following claims.

What is claimed is:

1. An apparatus for analyzing material comprising:
at least a first member and a second member movable with respect to each other;
at least one perfusion chamber, at least a portion of the at least one perfusion chamber in the first member and at least one portion of the at least one perfusion chamber in the second member;
at least one loading channel at least in the first member corresponding to the at least one perfusion chamber;
the first member and the second member movable with respect to each other between at least a first position and a second position, the first position such that the at least one perfusion chamber is formed by the portions of the at least one perfusion chamber in the first member and the second member being in communication with each other, and the second position such that the at least one loading channel is in communication with the portion of the at least one perfusion chamber in the second member.

2. The apparatus of claim 1, additionally comprising at least one flow channel formed in the first member and the second member, the at least one flow channel including the at least one perfusion chamber.

3. The apparatus of claim 2, additionally comprising an optical analysis system at least proximate to the at least one perfusion chamber.

4. The apparatus of claim 3, wherein the optical analysis system is in the second member.

5. The apparatus of claim 4, wherein the optical analysis system includes at least one reflector.

6. The apparatus of claim 5, wherein the optical analysis system includes at least one optical access port for supporting at least one optical fiber, the at least one optical access port positioned with respect to the at least one reflector for facilitating optical measurements across the at least one perfusion chamber in a direction at least substantially perpendicular to the longitudinal axis of the at least one perfusion chamber.

7. The apparatus of claim 6, wherein the optical access port is configured for accommodating optical fibers, and the optical fibers and the at least one reflector are configured for making absorbance measurements for media in the at least one perfusion chamber.

8. The apparatus of claim 6, wherein the optical access port is configured for accommodating optical fibers, and the optical fibers and the at least one reflector are configured for making fluorescence measurements for media in the at least one perfusion chamber.

9. The apparatus of claim 6, additionally comprising an inflow port in the second member in communication with the flow channel and an outflow port in the first member in communication with the flow channel.

10. The apparatus of claim 9, additionally comprising at least one oxygen sensor in communication with the flow channel between the at least one perfusion chamber and the outflow port.

11. The apparatus of claim 10, additionally comprising at least one oxygen sensor in communication with the flow channel between the inflow port and the at least one perfusion chamber.

12. The apparatus of claim 10, wherein the first member includes at least one port for supporting at least one optical fiber at least proximate to the at least one oxygen sensor in the flow channel intermediate the at least one perfusion chamber and the outflow port.

13. The apparatus of claim 12, wherein the first member and the second members are of a clear material.

14. The apparatus of claim 13, wherein the clear material is a transparent material.

15. The apparatus of claim 11, wherein the second member includes at least one port for supporting at least one optical fiber at least proximate to the at least one oxygen sensor in the flow channel intermediate the at least one perfusion chamber and the edge of the inflow port.

16. The apparatus of claim 15, wherein the second member is of a clear material.

17. The apparatus of claim 16, wherein the clear material is a transparent material.

18. The apparatus of claim 11, additionally comprising a pump for moving media through the at least one flow channel.

19. The apparatus of claim 18, wherein the pump is configured for moving the media either continuously or intermittently.

20. The apparatus of claim 11, additionally comprising at least one flow restrictor for the at least one flow channel intermediate the inflow port and the at least one perfusion chamber.

21. The apparatus of claim 11, wherein the at least one perfusion chamber and the at least one flow channel includes a plurality perfusion chambers each within a flow channel of a corresponding plurality of flow channels, and a plurality of loading channels, each loading channel of the plurality of loading channels corresponding to a flow channel and a corresponding perfusion chamber.

22. An apparatus for analyzing material comprising:
at least a first member and a second member movable with respect to each other between a first position and a second position;
an inflow port in the second member;
a plurality of outflow ports in the first member;
a plurality of flow channels, each flow channel of the plurality of flow channels in communication with the inflow port and an outflow port of the plurality of outflow ports;
a perfusion chamber formed in each of the flow channels of the plurality of flow channels, at least a portion of each perfusion chamber in the first member and the second member, the portions of each perfusion chamber in the first member and the second member enclosing a volume when the first member is in the first position with respect to the second member; and;
a plurality of loading channels in the first member, each of the loading channels of the plurality of loading channels corresponding to the perfusion chamber in each of the flow channels, the loading channels for communication with the portion of each perfusion chamber in the second member, when the first member is in the second position with respect to the second member.

23. The apparatus of claim 22, additionally comprising an optical system associated with each perfusion chamber.

24. The apparatus of claim 23, wherein the optical system includes at least one reflector proximate to a first side of the perfusion chamber and an optical access port extending into the apparatus for supporting at least one optical fiber, the optical access port positioned on an oppositely disposed second side of the perfusion chamber, and oriented with respect to the reflector such that optical measurements are taken at least substantially perpendicular to the orientation of the perfusion chamber.

25. The apparatus of claim 24, wherein the at least one optical fiber includes a plurality of optical fibers configured for making optical measurements selected from the group consisting of absorbance measurements and fluorescence measurements.

26. The apparatus of claim 24, wherein the one reflector is configured for reflecting light for at least one of absorbance measurements or fluorescence measurements.

27. The apparatus of claim 26, additionally comprising an inflow conduit in communication with the inflow port and each of the plurality of flow channels.

28. The apparatus of claim 27, additionally comprising a plurality of bores extending into the apparatus for accommodating at least one optical fiber associated with oxygen detection.

29. The apparatus of claim 28, wherein the plurality of bores includes a first bore proximate to the inflow conduit and a plurality of second bores, corresponding to each of the flow channels, proximate to each of the flow channels intermediate the perfusion chamber and the outflow port.

30. The apparatus of claim 29, additionally comprising, a plurality of oxygen sensors, the plurality of oxygen sensors including a first oxygen sensor in the inflow conduit proximate the first bore, and second oxygen sensors in each of the flow channels intermediate the perfusion chamber and the outflow port proximate to each of the second bores.

31. The apparatus of claim 29, additionally comprising at least one flow restrictor for each flow channel of the plurality of flow channels intermediate the perfusion chamber and the inflow port.

32. The apparatus of claim 29, additionally comprising at least one confinement member in each of the perfusion chambers.

33. The apparatus of claim 32, wherein the at least one confinement member includes two confinement members disposed at opposite ends of each perfusion chamber.

34. The apparatus of claim 22, wherein the first member and the second member include a clear material.

35. The apparatus of claim 34, wherein the clear material is a transparent material.

36. A method of material analysis comprising:
providing an apparatus comprising:
at least a first member and a second member movable with respect to each other;
at least one inflow port;
at least one outflow port;
at least one flow channel in communication with the at least one inflow port and the at least one outflow port;
at least one perfusion chamber in the at least one flow channel, at least a portion of the at least one perfusion chamber in the first member and at least one portion of the at least one perfusion chamber in the second member;
at least one loading channel at least in the first member corresponding to the at least one perfusion chamber;
the first member and the second member movable with respect to each other between at least a first position and a second position, the first position such that the at least one perfusion chamber is formed by the portions of the at least one perfusion chamber in the first member and the second member being in communication with each other so as to enclose a volume, and the second position such that the at least one loading channel is in communication with the portion of the at least one perfusion chamber in the second member;
moving the first member and the second member to the second position;
loading material into the perfusion chamber; and,
moving the first member and the second member to the first position, such that the material is enclosed in the volume of the at least one perfusion chamber.

37. The method of claim 36, wherein the material includes cells.

38. The method of claim 37, wherein perfusion media is moved through the at least one flow channel from the inflow port to the outflow port to perfuse the cells in the at least one perfusion chamber.

39. The method of claim 38, additionally comprising, performing at least one optical analysis on the cells in the at least one perfusion chamber.

40. The method of claim 39, wherein the at least one optical analysis includes making absorbance measurements.

41. The method of claim 40, additionally comprising, determining cytochrome-c amounts in the cells based on the absorbance measurements.

42. The method of claim 39, wherein the at least one optical analysis includes making fluorescence measurements.

43. The method of claim 42, additionally comprising, determining nicotinamide adenine dinucleotide (NAD+ and NADH) in the cells based on the fluorescence measurements.

44. The method of claim 39, wherein the optical analysis is performed by transmitting light energy at least substantially perpendicular to the vertical orientation of the at least one perfusion chamber.

45. The method of claim 38, additionally comprising, obtaining the oxygen concentration in the perfusion media.

46. The method of claim 45, wherein obtaining the oxygen concentration in the perfusion media includes sensing oxygen concentrations in the perfusion media intermediate the inflow port and the at least one perfusion chamber and intermediate the at least one perfusion chamber and the outflow port.

47. The method of claim 46, additionally comprising, determining the oxygen consumption in the cells from the sensed oxygen concentrations.
